SEQUENCE LISTING (9 sequences)

SEQ ID NO: 1 - DNA, 20 bp, synthetic construct
  source 1..20: mol_type = other DNA
ttgacctcagcgctgagttg

SEQ ID NO: 2 - DNA, 20 bp, synthetic construct
  source 1..20: mol_type = other DNA
cctgtagcccacgtcgtagc

SEQ ID NO: 3 - DNA, 18 bp, synthetic construct
  source 1..18: mol_type = other DNA
ccgaagtcatagccacac

SEQ ID NO: 4 - DNA, 18 bp, synthetic construct
  source 1..18: mol_type = other DNA
gtgccatcagagcagtct

SEQ ID NO: 5 - DNA, 24 bp, synthetic construct
  source 1..24: mol_type = other DNA
agaatatctttcaggacccctgct

SEQ ID NO: 6 - DNA, 23 bp, synthetic construct
  source 1..23: mol_type = other DNA
acaccagtgctgagcatactttt

SEQ ID NO: 7 - DNA, 21 bp, synthetic construct
  source 1..21: mol_type = other DNA
atgcctgaagaccctgccaag

SEQ ID NO: 8 - DNA, 21 bp, synthetic construct
  source 1..21: mol_type = other DNA
ggtcagttagccttgcctttg

SEQ ID NO: 9 - DNA, 26 bp, synthetic construct
  source 1..26: mol_type = other DNA
catgttccagtatgactccactcacg

(19) United States
(12) Patent Application Publication
(10) Pub. No.: US 2024/0382545 A1
(43) Pub. Date: Nov. 21, 2024

(54) COMPOSITION FOR PREVENTING, AMELIORATING OR TREATING RESPIRATORY DISEASE COMPRISING FERMENTED CENTELLA ASIATICA EXTRACT AS EFFECTIVE COMPONENT

(71) Applicant: KOREA INSTITUTE OF ORIENTAL MEDICINE, Daejeon (KR)

(72) Inventors: DONG-SEON KIM, Daejeon (KR); YOON-YOUNG SUNG, Daejeon (KR); EUN JUNG SON, Sejong-si (KR); YUN MI LEE, Daejeon (KR); HEUNG JOO YUK, Daejeon (KR)

(21) Appl. No.: 18/578,998

(22) PCT Filed: Jul. 12, 2022
(86) PCT No.: PCT/KR2022/010100

§ 371 Date: Jan. 12, 2024

(30) Foreign Application Priority Data
Jul. 14, 2021 (KR) 10-2021-0092393

(51) Int. Cl. A61K 36/23; A61K 9/00; A61K 35/744; A61K 35/747; A61K 36/06; A61P 11/00

(52) U.S. Cl. CPC A61K 36/23; A61K 9/0053; A61K 35/744; A61K 35/747; A61K 36/06; A61P 11/00

(57) ABSTRACT

A composition for preventing, ameliorating or treating a respiratory disease includes fermented Centella asiatica extract as effective component. The composition is effective in, in bronchial epithelial cells as respiratory cells, reducing the production amount of the inflammatory chemokine RANTES, protecting damaged cells induced by LPS, and reducing the expression level of the inflammatory cytokine IL-6, and it can also reduce the neutrophil count and regulate the expression level of inflammatory cytokines or chemokines in an animal model of lung injury. Accordingly, it can be advantageously used for a functional health food for preventing or ameliorating a respiratory disease, or a pharmaceutical product for preventing or treating a respiratory disease.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No PCT/KR2022/010100 filed on Jul. 12, 2022, which claims priority to the benefit of Korean Patent Application No. 10-2021-0092393 filed in the Korean Intellectual Property Office on Jul. 14, 2021, the entire contents of which are incorporated herein by reference.

### BACKGROUND

### 1. TECHNICAL FIELD

The present invention relates to a composition for preventing, ameliorating or treating a respiratory disease comprising fermented Centella asiatica extract as effective component.

### 2. BACKGROUND ART

Asthma, chronic obstructive pulmonary disease (COPD), allergic rhinitis, phlegm cough, acute and chronic bronchitis, bronchiolitis, laryngitis, pharyngitis, and tonsillitis are the representative examples of a respiratory disease. Asthma specifically refers to chronic inflammation occurring in the airways, especially in the bronchi. Inflammation triggered by asthma can be aggravated by various factors such as smoke, allergic antigens, cold air, physical exercise, respiratory infections, and more. Persistent inflammation leads to structural changes and hyper-responsiveness of the airways. As caused by these factors, symptoms such as wheezing (i.e., symptom showing a whistling or rattling breath sound caused by narrowed airways), shortness of breath, coughing, and excessive phlegm production are commonly exhibited.

The respiratory airway consists primarily of mucous membranes and muscles called bronchial smooth muscles. The mucous membranes contain numerous glands that continuously secrete necessary fluids. When the smooth muscles of the bronchi contract, the airway is narrowed. Once the inflammation is caused by various factors such as smoke, allergic antigens, cold air, physical exercise, and respiratory infections, an increased secretion from the glands is triggered, causing the fluid to block the airway and swelling of the mucous membranes toward the inside of the airway, consequently narrowing it further. As a result, sudden attacks of coughing accompanied by wheezing and severe difficulty in breathing occur. During an attack, dry coughing may occur and a sensation of chest tightness is often felt. There are also many cases of asthma caused by unknown factors showing just a difficulty in breathing with chronic coughing and chest tightness without wheezing, and these symptoms tend to occur suddenly in everyday life.

Meanwhile, Centella asiatica, commonly known as Byung-pool in South Korea, is a perennial plant frequently found in the mountains and fields of the southern islands of

Korean Peninsula. Its main stem spreads horizontally, and near the nodes where the roots descend, there are two degenerate scale-like leaves. The leaves densely grow from the nodes and are kidney-shaped, 2 to 5 cm in diameter, shiny on the surface, with blunt teeth along the edges. The length of the petiole is 4 to 20 cm. The flowers are reddish-purple, arranged in umbel clusters of 3 to 4 in the leaf axils, forming a head-like structure. The staminate flowers have 2 branches, and the fruit has a split and oblate shape. In India, Centella asiatica is called “tiger herb” because it is observed that tigers roll in areas with a lot of Centella asiatica to treat their wounds. People in India have used Centella asiatica as medicine for a long time. It has been discovered that the compound called madecassic acid present in the leaves and stems of Centella asiatica has anti-inflammatory properties and promotes the healing of tumors and wounds such as ulcers.

Due to these properties, Centella asiatica is used as a raw material for ointments, toothpaste, cosmetics, and other products.

Regarding the technologies related to Centella asiatica, in Korean Patent Application Publication No. 2003-0024932, a cosmetic composition containing Centella asiatica extract is disclosed. Additionally, in Korean Patent Registration No. 1591872, a cosmetic composition containing a Ge-8 mixture with excellent anti-inflammatory, anti-allergic, and antioxidant effects has been disclosed. However, as of now, there has been no disclosure of a composition for preventing, ameliorating or treating a respiratory disease comprising fermented Centella asiatica extract as effective component as described in the present invention.

### SUMMARY

The present invention is devised under the aforementioned circumstances, and it provides a composition for preventing, ameliorating or treating a respiratory disease comprising fermented Centella asiatica extract as effective component. It was found that the effective component of the present invention has an effect of reducing the production amount of the inflammatory chemokine RANTES, protecting damaged cells induced by LPS, and reducing the expression level of the inflammatory cytokine IL-6 in bronchial epithelial cells as respiratory cells, and also can reduce the neutrophil count and regulate the expression level of inflammatory cytokines or chemokines in an animal model of lung injury, and the present invention is completed accordingly.

To achieve the object described in the above, the present invention provides a functional health food composition for preventing or ameliorating a respiratory disease comprising fermented Centella asiatica extract as effective component.

The present invention further provides a pharmaceutical composition for preventing or treating a respiratory disease comprising fermented Centella asiatica extract as effective component.

The present invention further provides an animal feed additive for preventing or ameliorating a respiratory disease comprising fermented Centella asiatica extract as effective component.

The present invention still further provides a veterinary composition for preventing or treating a respiratory disease comprising fermented Centella asiatica extract as effective component.

The present invention relates to a composition for preventing, ameliorating or treating a respiratory disease comprising fermented Centella asiatica extract as effective component. Specifically, the effective component of the present invention is effective in, in bronchial epithelial cells as respiratory cells, reducing the production amount of the inflammatory chemokine RANTES, protecting damaged cells induced by LPS, and reducing the expression level of the inflammatory cytokine IL-6, and it can also reduce the neutrophil count and regulate the expression level of inflammatory cytokines or chemokines in an animal model of lung injury.

### BRIEF DESCRIPTION OF THE DRAWINGS

FIG. 1 demonstrates the anti-inflammatory effect determined by the inhibition of RANTES production in bronchial epithelial cells following a treatment with the fermented Centella asiatica extract of the present invention. *. **, and *** indicate a statistically significant decrease in RANTES production amount in the group treated with the fermented Centella asiatica extract of the present invention compared to the TNF-α treatment group. Specifically, * indicates p<0.05, ** indicates p<0.01, and *** indicates p<0.001.

FIG. 2 shows the results of determining cell viability following a treatment of bronchial epithelial cells with the fermented Centella asiatica extract of the present invention.

FIG. 3 shows the results of determining the cell-protective effect of the fermented Centella asiatica extract of the resent invention in bronchial epithelial cells which have been previously damaged by LPS treatment. #indicates a statistically significant decrease in cell viability in the LPS treatment group compared to the untreated control group (CON), with p<0.05. Additionally, * indicates a statistically significant increase in cell viability in the group treated with the fermented Centella asiatica extract of the present invention compared to the LPS treatment group, with p<0.05.

FIG. 4 shows the results of determining the changes in the expression level of the inflammatory cytokine IL-6 in bronchial epithelial cells (BEAS-2B cells) which have been first treated with LPS to cause inflammation and then treated with the fermented Centella asiatica extract of the present invention. ### indicates a statistically significant increase in IL-6 expression level in the LPS treatment group compared to the untreated control group (CON), with p<0.001. ** indicates a statistically significant decrease in the expression level of IL-6 in the group treated with the Centella asiatica ethanol extract (CA2) and the group treated with the fermented Centella asiatica extract of the present invention (CA3 to CA5) compared to the LPS treatment group, with p<0.01. $ and $$ indicate a statistically more significant decrease in the expression level of IL-6 in the group treated with the fermented Centella asiatica extract of the present invention (CA4and CA5) compared to the group treated with the Centella asiatica ethanol extract (CA2), with p<0.05 for $ and p<0.01 for $$.

FIGS. 5A to 5E show the results the Diff-Quick staining for determining, in the animal model of lung injury, the total neutrophil count in the bronchoalveolar lavage fluid (BALF) isolated from the group treated with the lactic acid bacteria-fermented Centella asiatica extract of the present invention. In the FIGS. 5A to 5E, FIG. 5A represents the normal group, FIG. 5B represents the control group with lung injury, FIG. 5C represents the group treated with 100 mg/kg of lactic acid bacteria-fermented Centella asiatica extract, FIG. 5D represents the group treated with 200 mg/kg of lactic-acid bacteria-fermented Centella asiatica extract, and FIG. 5E represents the group treated with dexamethasone, which is a positive control.

FIG. 6 is a graph showing the results of determining, in the animal model of lung injury, the total neutrophil cell count in the bronchoalveolar lavage fluid (BALF) isolated from the group treated with the lactic acid bacteria-fermented Centella asiatica extract of the present invention. ### indicates a statistically significant increase in neutrophil cell count in the control group (COPD_CTL) compared to the normal group (Balb/c Normal), with p<0.001. *** indicates a statistically significant decrease in neutrophil cell count in the group administered with the lactic acid bacteria-fermented Centella asiatica extract of the present invention (COPD_CA4) compared to the control group, with p<0.001.

FIG. 7 shows the results of determining, in the animal model of lung injury, the changes in the production amounts of chemokines and cytokines (MIP2, IL-1α, CXCL-1, IL-17, and TNF-α) in bronchoalveolar lavage fluid (BALF) isolated from the groups administered with the lactic acid bacteria-fermented Centella asiatica extract of the present invention, in which the changes were followed by ELISA. ## and ### indicate a statistically significant increase in the production amounts of MIP2, IL-1α, CXCL-1, IL-17, and TNF-α in the control group with lung injury (COPD_CTL) compared to the normal group (Balb/c Normal), with ##representing p<0.01 and ### representing p<0.001. * and ** indicate a statistically significant decrease in the production amounts of MIP2, IL-1α, CXCL-1, IL-17, and TNF-60 in the positive control group and the group administered with the lactic acid bacteria-fermented Centella asiatica extract of the present invention compared to the control group with lung injury, with * representing p<0.05 and ** representing p<0.01.

FIG. 8 shows the results of determining, in the animal model of lung injury, the changes in the gene expression level of chemokines and cytokines (MUC5AC, TNF-α, CXCL-1, and MIP2) in bronchoalveolar lavage fluid (BALF) isolated from the groups administered with the lactic acid bacteria-fermented Centella asiatica extract of the present invention, in which the changes were followed by ELISA. ##and ###indicate a statistically significant increase in the gene expression levels of MUC5AC, TNF-α, CXCL-1, and MIP2 in the control group with lung injury (COPD_CTL) compared to the normal group (Balb/c Normal), with ##representing p<0.01 and ###representing p<0.001. * and ** indicate a statistically significant decrease in the gene expression levels of MUCSAC, TNF-α, CXCL-1, and MIP2 in the positive control group and the group administered with the lactic acid bacteria-fermented Centella asiatica extract of the present invention compared to the control group with lung injury, with * representing p<0.05 and ** representing p<0.01.

### DETAILED DESCRIPTION

The present invention relates to a functional health food composition for preventing or ameliorating a respiratory disease comprising fermented Centella asiatica extract as effective component.

The respiratory disease is preferably selected from the group consisting of asthma, chronic obstructive pulmonary disease, bronchitis, pharyngitis, tonsillitis, and laryngitis, but it is not limited to them.

The fermented Centella asiatica extract can be produced by a method including the following steps:
1) adding water to Centella asiatica powder followed by sterilizing:
2) after the step 1) above, cooling to room temperature and adding fermentation strain followed by fermentation at 25 to 50° C. to prepare a fermented product of Centella asiatica; and
3) adding an extraction solvent to the fermented product of Centella asiatica of the step 2) above followed by extracting,
but it is not limited thereto.

In the above step 2), the room temperature is preferably between 15 and 25° C., but it is not limited thereto. The time for preparing the fermented product is preferably 2 to 4 days, but it is not limited thereto.

The fermentation strain added in step 2) above is preferably lactic acid bacteria, yeast, or koji (i.e., Aspergillus oryzae), but it is not limited thereto. The lactic acid bacteria in this context are preferably one or more selected from Enterococcus faecium, Lactobacillus casei, Lactobacillus acidophilus, Lactobacillus plantarum, Leuconostoc kimchii, Leuconostoc citreum, Leuconostoc mesenteroides, and Lactobacillus sakei. More preferably, they are a combination of lactic acid bacteria consisting of Lactobacillus plantarum, Leuconostoc kimchii, Leuconostoc citreum, Leuconostoc mesenteroides, and Lactobacillus sakei, but not limited thereto.

The extraction solvent of the above step 3) is preferably selected from water, C₁-C₄ lower alcohol, and a mixture thereof. It is more preferably ethanol, but not limited thereto. With regard to the production method described in the present invention, any kind of common methods that are generally known as extraction method in the pertinent art, e.g., filtration, hot water extraction, impregnation extraction, extraction by reflux condensation, and ultrasonic extraction, can be used. It is preferable that the extraction is carried out by adding the extraction solvent in an amount of 1 to 20 times the weight of fermented Centella asiatica. More preferably, the extraction solvent is added in an amount of 10 to 18 times the weight of fermented Centella asiatica. Even more preferably, the extraction solvent is added in an amount of 15 times the weight of fermented Centella asiatica. The extraction temperature is preferably between 20° C. and 120° C., but it is not limited thereto. Furthermore, the extraction time is preferably between 1 hour and 48 hours, more preferably between 1 hour and 24 hours, and most preferably 2 hours, but it is not limited thereto. It is preferable that the concentration under reduced pressure of the step (3) in the above method is preferably carried out by using a vacuum concentration apparatus or a vacuum rotary evaporator, but it is not limited thereto. Additionally, the drying can be preferably performed by drying under reduced pressure, drying under vacuum, drying under boiling, spray drying, or freeze-drying, and more preferably freeze-drying, but it is not limited thereto.

The composition is preferably prepared in any one formulation selected from powder, granule, pill, tablet, capsule, candy, syrup, and beverage, but it is not limited thereto.

The functional health food composition of the present invention can be prepared by adding the fermented Centella asiatica extract by itself or mixing it with other food product or other food component. The composition can be suitably prepared by following a common method. Examples of the food product to which the fermented Centella asiatica extract of the present invention can be added include caramel, meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. In other words, the type of the food products is not particularly limited. The functional health food composition may further comprise various nutritional supplements, a vitamin, a mineral (i.e., electrolyte), a synthetic or natural flavor, a coloring agent, an enhancer (i.e., cheese, chocolate, etc.), pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated beverage. Other than those, fruit pulp for producing fruit juice or vegetable beverage may be additionally comprised. Those components may be used either independently or in combination thereof. The functional health food composition of the present invention may further include various flavorings or natural carbohydrates as additional ingredients. The natural carbohydrates may include monosaccharides such as glucose and fructose, disaccharides such as maltose and sucrose, and polysaccharides such as dextrin and cyclodextrin, as well as sugar alcohols such as xylitol, sorbitol, and erythritol. The ratio of the natural carbohydrates is, although not critically important, preferably about 0.01 to 0.04 g, more preferably about 0.02 to 0.03 g per 100 g of the composition of the present invention, but it is not limited thereto. As sweeteners, natural sweeteners such as thaumatin and stevia extracts, or synthetic sweeteners such as saccharin and aspartame, can be used.

The present invention further relates to a pharmaceutical composition for preventing or treating a respiratory disease comprising fermented Centella asiatica extract as effective component.

The pharmaceutical composition of the present invention may further comprise a pharmaceutically acceptable carrier, excipient, or diluent. Pharmaceutically acceptable carrier comprised in the pharmaceutical composition of the present invention is a carrier commonly used in preparing formulations, such as saline, sterile water, Ringer's solution, buffered saline, dextrose solution, maltodextrin solution, glycerol, ethanol, lactose, dextrose, sucrose, sorbitol, mannitol, starch, acacia gum, calcium phosphate, alginate, gelatin, calcium carbonate, microcrystalline cellulose, polyvinylpyrrolidone, cellulose, syrup, methyl cellulose, methyl hydroxybenzoate, propyl hydroxybenzoate, talc, magnesium stearate, and mineral oil, but not limited thereto. Additionally, besides the aforementioned ingredients, the pharmaceutical composition may further comprise antioxidants, buffers, stabilizers, diluents, surfactants, binders, lubricants, moistening agents, sweeteners, flavoring agents, emulsifiers, suspending agents, or preservatives. The pharmaceutical composition of the present invention can be administered either orally or parenterally, in which parenteral administration can be done by injection or by application onto the skin. When applied onto the skin, it is preferable to use formulations such as ointments, patches, or sprays. The appropriate dosage of the pharmaceutical composition of the present invention may vary depending on factors such as the method of formulation, mode of administration, patient's age, weight, gender, pathological condition, diet, administration time, route of administration, excretion rate, and responsiveness to treatment.

The present invention further relates to an animal feed additive for preventing or ameliorating a respiratory disease comprising fermented Centella asiatica extract as effective component.

The animal feed additive of the present invention corresponds to a supplementary animal feed in terms of the animal feed management regulations. In the present invention, the term ‘animal feed’ may refer to any natural or artificial prescribed feed, single feed, or the components of such single feed, which animals eat, ingest, and digest. The types of the animal feed are not particularly limited and can include the feeds that are commonly used in the relevant technical field. Non-limiting examples of such feeds include plant-based feeds such as grains, root vegetables, by-products of food processing, poultry, fibers, pharmaceutical by-products, oils, starches, gourd proteins, and cereal by-products; and animal-based feeds such as proteins, minerals, oils, mineral substances, fats, single-cell proteins, animal plankton, or food. These can be used either alone or in a mixture of two or more of them.

The present invention still further relates to a veterinary composition for preventing or treating a respiratory disease comprising fermented Centella asiatica extract as effective component.

The veterinary composition of the present invention may further comprise appropriate excipients and diluents according to conventional methods. Examples of the excipients and diluents that can be comprised in the veterinary composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinylpyrrolidone, water, methylhydroxy benzoate, propy lhydroxy benzoate, talc, magnesium stearate, cetanol, liquid paraffin, sorbitan monostearate, polysorbate 60, methylparaben, propylparaben, and mineral oil. The veterinary composition according to the present invention may additionally include fillers, anticoagulants, lubricants, moistening agents, flavorings, emulsifiers, preservatives, and the like. The veterinary composition according to the present invention can be formulated using well-known methods in the industry to provide rapid, sustained, or delayed release of the active ingredient after administration to animals. The formulation can take various forms such as powder, granule, tablet, capsule, suspension, emulsion, solution, syrup, aerosol, soft or hard gelatin capsule, suppository, sterile injectable solution, or sterile topical preparation.

The effective amount of the veterinary composition according to the present invention can be suitably selected according to the individual characteristics of the animal. Specifically, the effective amount can be determined based on factors such as the severity of the disease or condition, the age, weight, health status, or gender of the animal, sensitivity to the effective component of the present invention, the route of administration, the duration of administration, other composition used in combination or simultaneously with the composition of the present invention, and other factors that are well known in the fields of physiology and veterinary medicine.

Hereinbelow, the present invention is explained in greater detail in view of Examples. However, the following Examples are given only for more specific explanation of the present invention and it is evident to a person who has common knowledge in the pertinent art that the scope of the present invention is not limited by them.

### EXAMPLES

Example 1. Preparation of fermented Centella asiatica extract, Centella asiatica extract, and fermented product of Centella asiatica extract

(1) Preparation of ethanol extract of Centella asiatica after fermentation (by lactic acid bacteria, koji, or yeast)

Lactic acid bacteria used for the fermentation were a combination of lactic acid bacteria, which consists of Lactobacillus plantarum, Leuconostoc kimchii, Leuconostoc citreum, Leuconostoc mesenteroides, and Lactobacillus sakei. Sterilization was carried out at 121° C. for 15 minutes after adding 400 ml of distilled water. After cooling to room temperature, 20 ml of the combination of the lactic acid bacteria, cultured in MRS broth at a concentration of 1×10⁴ cfu/ml, was added to the Centella asiatica obtained after the cooling to room temperature. Fermentation was carried at 32° C. for 3 days followed by drying at 60° C. to prepare the lactic acid bacteria-fermented product of Centella asiatica.

Additionally, other than the use of 10 g of koji or 2 g of yeast instead of the aforementioned combination of lactic acid bacteria, the same method was employed to prepare a koji-fermented product of Centella asiatica and a yeast-fermented product of Centella asiatica, respectively.

The koji-fermented product of Centella asiatica, lactic acid bacteria-fermented product of Centella asiatica, and yeast-fermented product of Centella asiatica were then extracted with 70% (v/v) ethanol to produce koji-fermented Centella asiatica extract (CA3), lactic acid-bacteria-fermented Centella asiatica extract (CA4), and yeast-fermented Centella asiatica extract (CA5), respectively.

(2) Water or ethanol extract of Centella asiatica

For the water extract of Centella asiatica (CA1), 200 g of Centella asiatica were ground, and 2 liters of water were added for hot water extraction. For the ethanol extract of Centella asiatica (CA2), 200 g of Centella asiatica were ground, and 2 liters of 70% (v/v) ethanol were added to obtain the ethanol extract of Centella asiatica. These extracts were used as a comparative example in the efficacy evaluation for respiratory disease.

(3) Preparation of ethanol extract of Centella asiatica and lactic acid bacteria, koji, or yeast-fermented product of ethanol extract of Centella asiatica

The ethanol extract of Centella asiatica obtained as mentioned above was subjected to concentration under reduced pressure. To the resulting concentrated extract, 500 ml of sterilized water were added. Then, 20 ml of a combination of lactic acid bacteria cultured in MRS broth at a concentration of 1×10⁴ cfu/ml were added. The mixture was cultured at 32° C. for 2 days. After the completion of the culture, the resultant was dried under reduced pressure at 60° C. to produce the lactic acid bacteria-fermented product of Centella asiatica extract (CA7). The combination of lactic acid bacteria used in the fermentation process is a combination of Lactobacillus plantarum, Leuconostoc kimchii, Leuconostoc citreum, Leuconostoc mesenteroides, and Lactobacillus sakei.

Additionally, during the fermentation process, other than the use of 10 g of koji or 2 g of yeast instead of the aforementioned combination of lactic acid bacteria, the same method was employed to prepare a koji-fermented product of Centella asiatica extract (CA6) and a yeast-fermented product of Centella asiatica extract (CA8), respectively.

Example 2. Determination of effect of improving inflammation induced by TNF-α in bronchial epithelial cells

The human bronchial epithelial cell line BEAS-2B (ATCC, USA) was cultured in Dulbecco's Modified Eagle's medium (DMEM) supplemented with fetal bovine serum (FBS) and penicillin-streptomycin (PS). The cells were seeded at a density of 5×10⁴ cells per well in a 96-well plate and cultured in DMEM medium for 18 hours. The medium was removed and replaced with serum-free DMEM, and the cells were treated with test sample (6.25, 12.5, 25, 50, 100, and 200 μg/ml of fermented Centella asiatica extract) along with simultaneous treatment with 10 ng/ml of TNF-α. The cells were then cultured for 24 hours. The amount of RANTES secretion present in cell culture supernatant was measured by using an ELISA kit (R&D Systems, USA) according to the manufacturer's instructions. Statistical significance was evaluated using the Student's t-test.

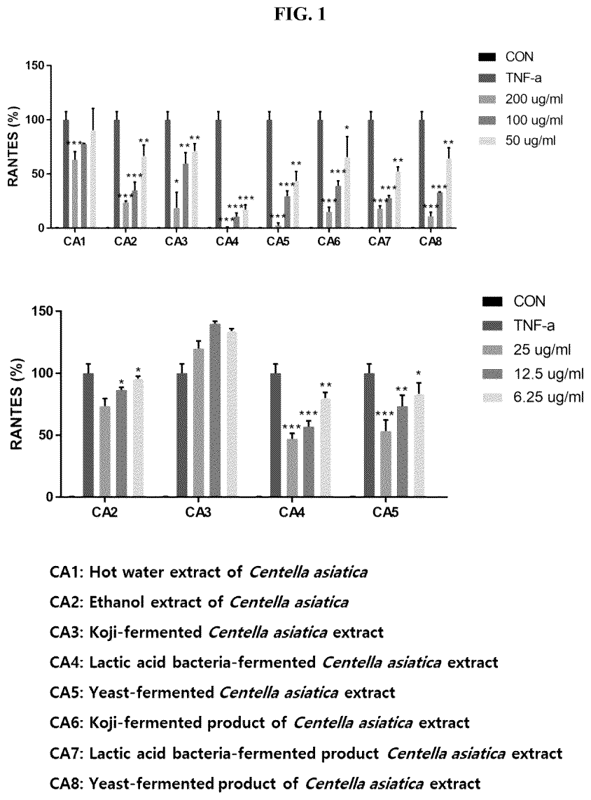

As indicated in FIG. 1, the results show that the treatment with TNF-α led to an increase in the amount of RANTES secretion. However, in contrast to such result, when treated with the fermented product of Centella asiatica extract (CA6, CA7, and CA8) of the present invention as well as the fermented Centella asiatica extract (CA3, CA4, and CA5), there was a statistically significant decrease in the amount of RANTES secretion.

Example 3. Determination of cell viability and effect of protecting cell damages induced by LPS in bronchial epithelial cells

(1) Evaluation of cell viability using human bronchial epithelial cell line

The human bronchial epithelial cell line BEAS-2B (ATCC, USA) was cultured in Dulbecco's Modified Eagle's medium (DMEM) supplemented with fetal bovine serum (FBS) and penicillin-streptomycin (PS). Cells were seeded at a density of 3×10⁴ cells per well in a 96-well plate and cultured in DMEM medium for 18 hours. The medium was removed and replaced with serum-free DMEM, and the cells were treated with 100 μg/ml of fermented Centella asiatica extract followed by culture for 24 hours. Cell viability was measured using the CCK-8 kit. Statistical significance was assessed using the Student's t-test.

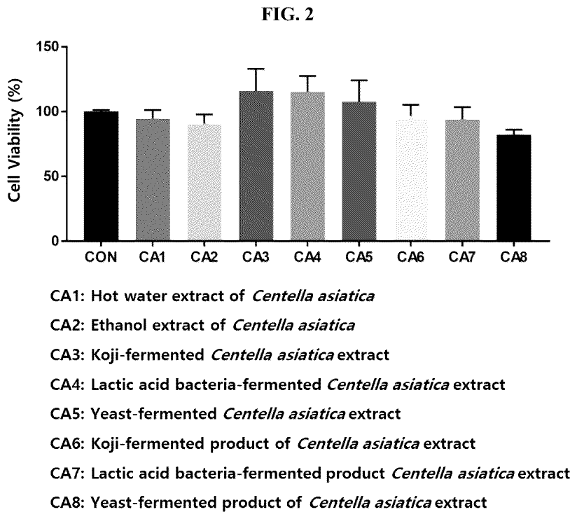

As shown in FIG. 2, the results indicate that the fermented Centella asiatica extract exhibited no cytotoxicity.

(2) Effect of protecting cell damages induced by LPS using human bronchial epithelial cell line

The human bronchial epithelial cell line BEAS-2B (ATCC, USA) was cultured in Dulbecco's Modified Eagle's medium (DMEM) supplemented with fetal bovine serum (FBS) and penicillin-streptomycin (PS). Cells were seeded at a density of 3×10⁴ cells per well in a 96-well plate and cultured in DMEM medium for 18 hours. The medium was removed and replaced with serum-free DMEM, and the cells were treated with 100 μg/ml of fermented Centella asiatica extract or 20 g/ml of LPS followed by culture for 24 hours. Cell viability was measured using the CCK-8 kit. Statistical significance was assessed using the Student's t-test.

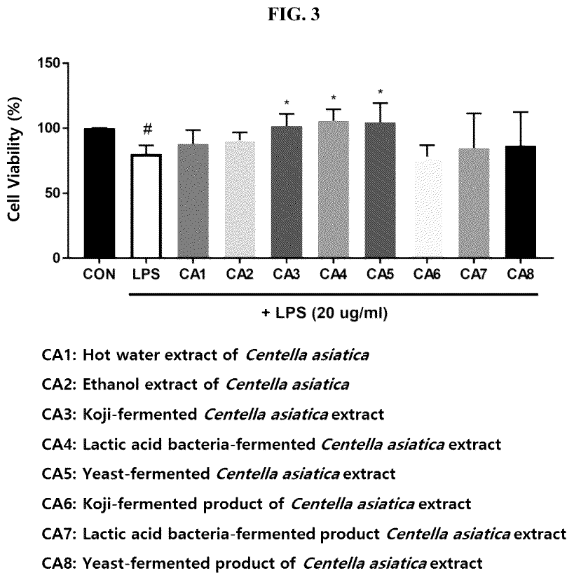

The results revealed that LPS treatment for causing cytotoxicity induced a decrease in cell viability. On the contrary, the fermented Centella asiatica extract of the present invention (CA3, CA4, and CA5) demonstrated an increase in cell viability compared to the LPS-treated group. Based on these findings, it was recognized that the fermented Centella asiatica extract of the present invention has a protective effect on cells (FIG. 3).

Example 4. Determination of changes in expression level of inflammatory cytokine IL-6 after inducing inflammation using LPS in bronchial epithelial cell BEAS-2B

Change in the expression level of the inflammatory cytokine IL-6 was assessed in human bronchial epithelial cell line BEAS-2B having inflammation induced by LPS.

BEAS-2B cells were cultured in Dulbecco's Modified Eagle's medium (DMEM) supplemented with fetal bovine serum (FBS) and penicillin-streptomycin (PS). BEAS-2B cells (3×10⁴ cells/well) were seeded in a 96-well plate with 10% FBS-containing DMEM and cultured for 18 hours. The medium was removed and replaced with serum-free DMEM, and the cells were treated with test sample (50, 25 μg/ml) or LPS (100 ng/ml) followed by culture for 24 hours. After 24 hours, the cell supernatant was collected, and the IL-6 expression level was measured using an IL-6 ELISA kit.

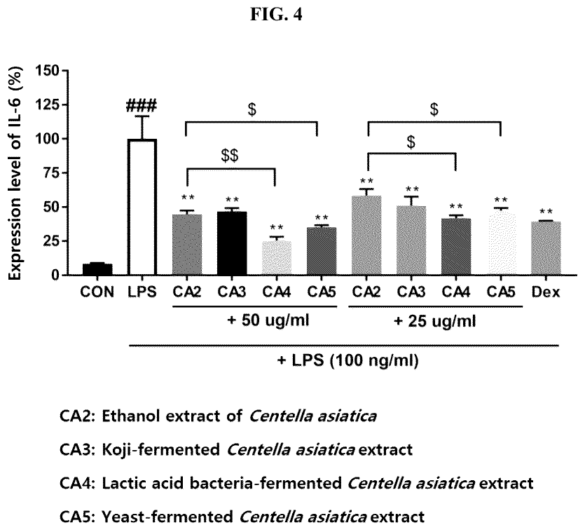

As depicted in FIG. 4, the results show a significant increase in IL-6 expression level in the LPS induction group compared to the control group (CON). On the contrary, there was a significant decrease in the IL-6 expression level in the groups treated with Centella asiatica extract (CA2) or fermented Centella asiatica extracts (CA3, CA4,and CA5) compared to the LPS induction group. Particularly noteworthy is that, compared to the Centella asiatica extract, the lactic acid bacteria-fermented Centella asiatica extract (CA4) and the yeast-fermented Centella asiatica extract (CA5) exhibited a more significant decrease in IL-6 expression level.

Example 5. Determination of effect of improving respiratory inflammation in animal model of lung injury

Over a period of 2 weeks, 7-week-old male BALB/c mice were induced with chronic obstructive pulmonary disease (COPD) by inhalation of a total of 100 μL of LPS+CS ((standard cigarette smoke extract), 50 μL each through the nose and mouth), three times in total. The model animals were randomly categorized into the following groups: a normal group (Normal) without any treatment, a control group (Control) treated with the COPD-inducing substance, a group treated with the COPD-inducing substance and 3 mg/kg of dexamethasone as a positive control, a group treated with the COPD-inducing substance and 200 mg/kg of lactic acid bacteria-fermented Centella asiatica extract (CA4-200), and a group treated with the COPD-inducing substance and 100 mg/kg of lactic acid bacteria-fermented Centella asiatica extract (CA4-100). The drug-administered groups (dexamethasone or lactic acid bacteria-fermented Centella asiatica extract) received the daily oral administration for 14 days. Upon the completion of the test, bronchoalveolar lavage fluid (BALF) and lung tissues were collected from the mice of each group.

(1) Isolation of bronchoalveolar lavage fluid (BALF) and measurement of neutrophil count

After collecting blood, the mice dissection was carried out and, for obtaining the total cell count in BALF, blood cells precipitated by centrifugation were separated and stained with 0.04% trypan blue, and then the total cell count was measured. Diff-Quick staining was performed three times, followed by two washes with PBS. Nine slides were then prepared for each group, and cell counting was performed at 400x magnification using an optical microscope.

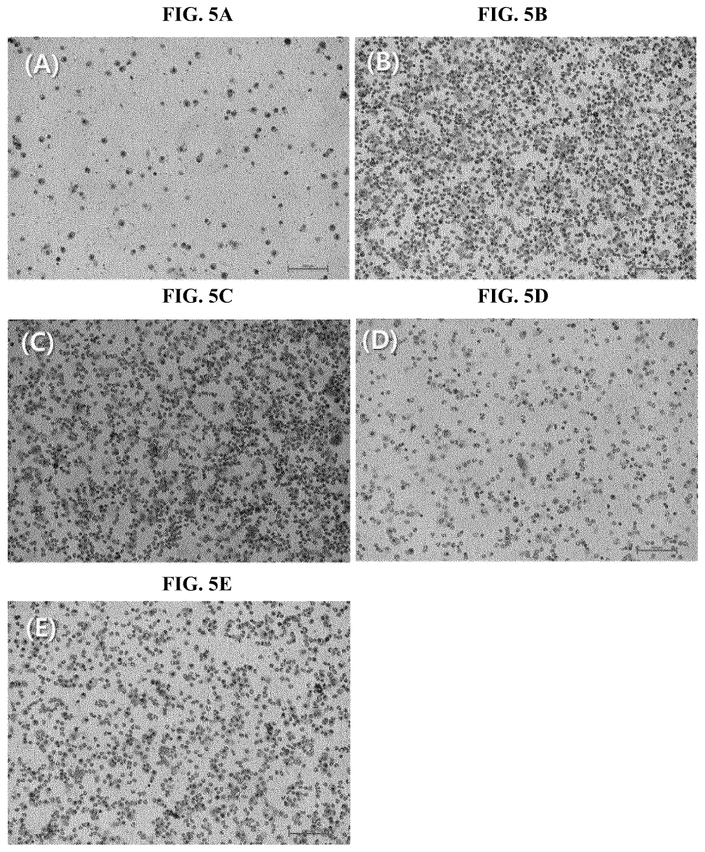

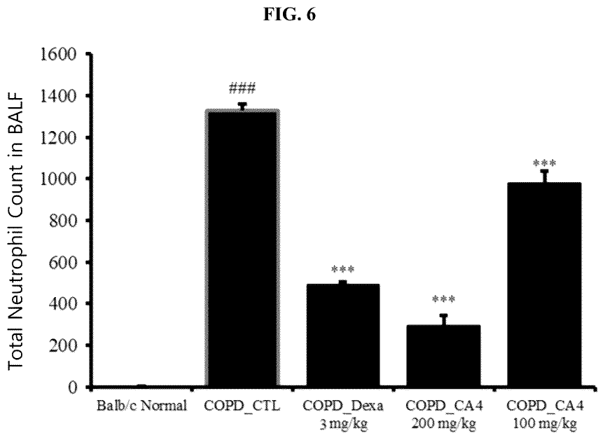

The results showed a decrease in the relative neutrophil count in the group treated with 3 mg/kg dexamethasone as a positive control and the group treated with 100 mg/kg or 200 mg/kg of lactic acid bacteria-fermented Centella asiatica extract (CA4) of the present invention. Particularly, it was found that the group treated with 200 mg/kg of lactic acid bacteria-fermented Centella asiatica extract exhibits the most significant decrease in neutrophil count (FIGS. 5A to 6).

(2) Enzyme-linked immunosorbent assay (ELISA)

ELISA was performed to measure the production amount of chemokines and cytokines (MIP2, IL-1α, CXCL-1, IL-17, and TNF-α) in bronchoalveolar lavage fluid (BALF) isolated from the aforementioned animal model (C57BL/6 mice). Capture antibody was mixed with coating buffer and added to each well at 100 μL per well. The plate was left overnight at 4° C., followed by four washes with washing buffer. Subsequently, 200 μL of assay diluent were added to each well, and the plate was blocked at room temperature for 1 hour. After four washes with washing buffer, serum and standard substances for each test group were diluted tenfold in assay diluent solution, and 100 μL of each dilution were added to the 96-well plate coated with respective capture antibody. The plate was incubated at room temperature for 2 hours. After three washes, biotin-conjugated antibody reagent was added to each well (100 μL per well) and the reaction was allowed to occur at room temperature for 1 hour, followed by two washes with washing buffer. Streptavidin-HRP solution was then added to each well (100 μL per well) and incubated at room temperature for 1 hour, followed by two washes with washing buffer. Then, 100 μL of substrate solution were added and the reaction was allowed to occur for 20 minutes. Finally, the reaction was terminated by adding 50 μL of stop solution, and the absorbance was measured at 450 nm.

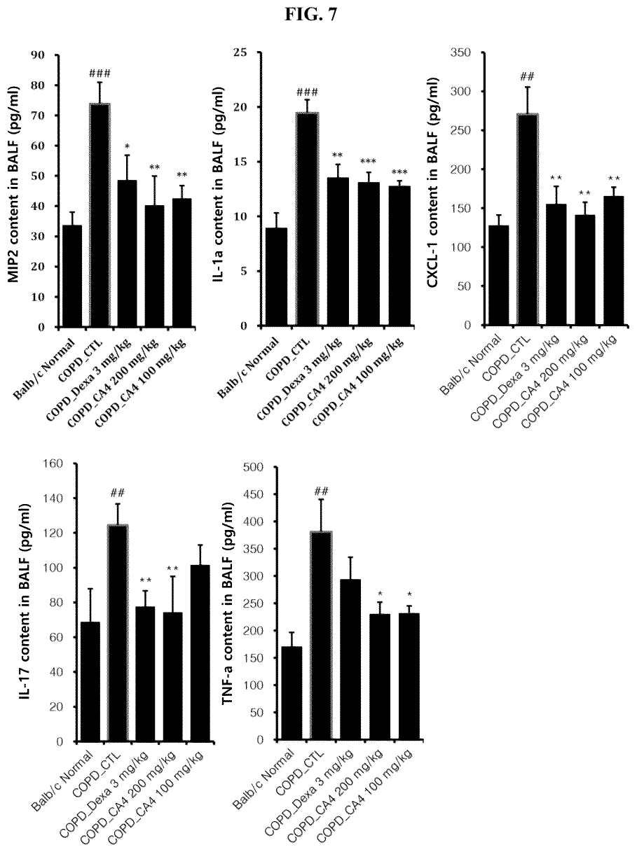

The results indicated that the inhalation of LPS+standard cigarette smoke extract in the control group led to an increase in the production amount of inflammatory chemokines and cytokines in BALF. On the contrary, a statistically significant decrease in the production amount of MIP2, IL-1α, CXCL-1, IL-17, and TNF-α was observed in the group treated with dexamethasone as a positive control and also the group treated with lactic acid bacteria-fermented Centella asiatica extract (CA4) (FIG. 7).

(3) Real-time polymerase chain reaction (Real-Time PCR)

Real-time polymerase chain reaction (PCR) using cDNAs of respiratory inflammatory factors including MUC5AC (Mucin-5AC), TNF-α, CXCL-1, and MIP2,which have been constructed using lung tissues, was conducted on the Applied Biosystems 7500 Real-Time PCR system (Applied Biosystems, USA). As a G3PDH (mouse glyceraldehyde-3-phosphate dehydrogenase) probe,

CATGTTCCAGTATGACTCCACTCACG (SEQ ID NO 9) (VIC) (i.e., the probe is a product provided by Applied Biosystems) was used, and Sper-TaqMan PCR master mix (4369016, ABI) was also used. The reaction was carried out after adjusting the final concentration of primers MUC5AC, TNF-α, CXCL-1, and MIP2 to 200 nM.

The conditions for real-time polymerase chain reaction were as follows: pre-denaturation at 50° C. for 2 minutes, followed by denaturation at 94° C. for 10 minutes, and then the amplification through 40 cycles including 95° C. for 0.15 minute and 60° C. for 1minute. For the group administered with the lactic acid bacteria-fermented Centella asiatica extract and Control, relative quantitative (RQ) was measured by using G3PDH as internal standard.

TABLE 1
Sequences of primers and probes used for real-time
PCR

| | | | SEQ |
|---|---|---|---|
| | | | ID |
| Name | Direction | Sequence (5′→3′) | NO: |
| TNF-α | Forward | TTG ACC TCA GCG CTG AGT TG | 1 |
| | Reverse | CCT GTA GCC CAC GTC GTAGC | 2 |
| CXCL1 | Forward | CCG AAG TCA TAG CCA CAC | 3 |
| | Reverse | GTG CCA TCA GAG CAG TCT | 4 |
| MUC5AC | Forward | AGA ATA TCT TTC AGG ACC CCT | 5 |
| | | GCT | |
| | Reverse | ACA CCA GTG CTG AGC ATA CTT | 6 |
| | | TT | |
| MIP-2 | Forward | ATG CCT GAA GAC CCT GCC AAG | 7 |
| | Reverse | GGT CAG TTA GCC TTG CCT TTG | 8 |
| G3PDH | | CAT GTT CCA GTA TGA CTC CAC | 9 |
| (probe) | | TCA CG | |

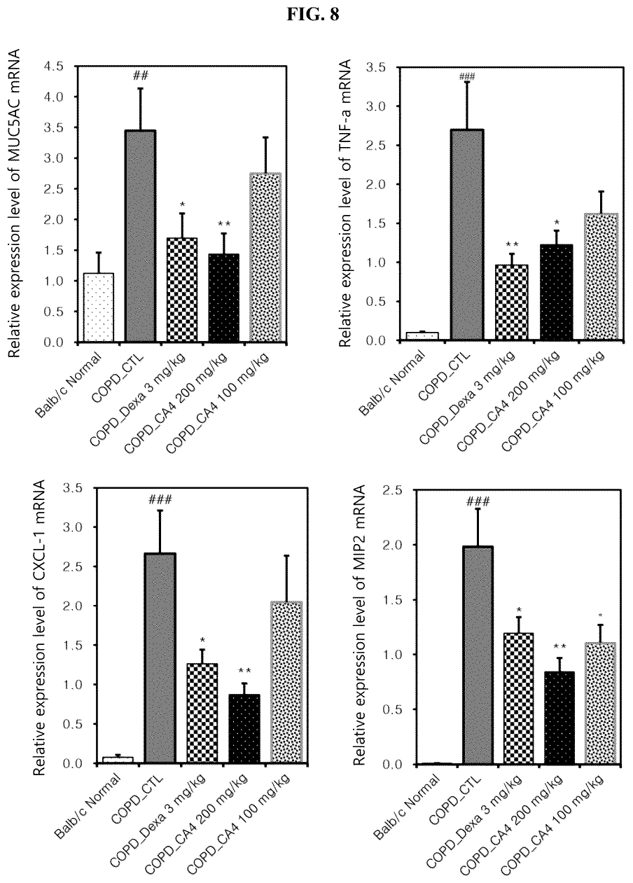

The results showed that, in the lungs of C57BL/6 mice subjected to inhalation of LPS+standard cigarette smoke extract as Control, there was an increase in the gene expression level of both inflammatory chemokines and cytokines (MUC5AC, TNF-α, CXCL-1, and MIP2). On the contrary, from the group treated with dexamethasone as a positive control and the group treated with the lactic acid bacteria-fermented Centella asiatica extract of the present invention (CA4), a decrease in the gene expression level of MUCSAC, TNFα, CXCL-1, and MIP2 was observed (FIG. 8).

### REFERENCE TO AN ELECTRONIC SEQUENCE LISTING

A sequence listing electronically submitted on Jan. 12, 2024 as a XML file named 20240112_S27323GR15_TU_SEQ.XML. created on Jan. 2, 2024. and having a size of 8.875 bytes. is incorporated herein by reference in its entirety.

1-11. (canceled)

12. A method for ameliorating or treating a respiratory disease, the method comprising administering a composition comprising fermented Centella asiatica extract as effective component to a subject in need thereof.

13. The method of claim 12, wherein the fermented Centella asiatica extract is produced by:
adding water to Centella asiatica powder;
sterilizing the Centella asiatica powder in the water;
cooling the sterilized Centella asiatica powder to room temperature and adding fermentation strain followed by fermentation at 25 to 40° C. to prepare a fermented product of Centella asiatica; and
adding an extraction solvent to the fermented product of Centella asiatica to produce the fermented Centella asiatica extract.

14. The method of claim 13, wherein the fermentation strain is lactic acid bacteria or yeast.

15. The method of claim 13, wherein the fermentation strain is lactic acid bacteria selected from the group consisting of Enterococcus faecium, Lactobacillus casei, Lactobacillus acidophilus, Lactobacillus plantarum, Leuconostoc kimchii, Leuconostoc citreum, Leuconostoc mesenteroides, and Lactobacillus sakei.

16. The method of claim 13, wherein the extraction solvent comprises water, C₁-C₄ lower alcohol, or a mixture thereof.

17. The method of claim 12, wherein the respiratory disease is one or more selected from the group consisting of asthma, chronic obstructive pulmonary disease, bronchitis, pharyngitis, tonsillitis, and laryngitis.

18. The method of claim 12, wherein the composition is prepared in any one formulation selected from powder, granule, pill, tablet, capsule, candy, syrup, and beverage.

19. The method of claim 12, wherein the composition is a pharmaceutical composition further comprising a pharmaceutically acceptable carrier, excipient, and/or diluent.

20. The method of claim 12, wherein the subject is a human.

21. The method of claim 12, wherein the subject is an animal.
